(19) United States
(12) Patent Application Publication
(10) Pub. No.: US 2008/0220064 A1
(43) Pub. Date: Sep. 11, 2008

(54) EXTENDED RELEASE MATRIX FORMULATIONS OF MORPHINE

(76) Inventors: Ketkar Anant RAMESH, Pune (IN); Pratik Kumar, Muzaffarpur (IN); Ashok Rampal, Amritsar (IN)

Correspondence Address: Jayadeep R. Deshmukh, Esq.;Ranbaxy Inc., Suite 2100, 600 College Road East, Princeton, NJ, 08540, US

(21) Appl. No.: 11/951,885

(22) Filed: Dec. 6, 2007

(30) Foreign Application Priority Data
Dec. 6, 2006 (IN) 2615/DEL/2006

(51) Int. Cl. A61K 9/00; A61K 31/485

(52) U.S. Cl. 424/485; 424/484; 514/282; 424/488

(57) ABSTRACT

The present invention provides extended-release matrix formulations comprising a therapeutically effective amount of morphine or salt thereof, one or more hydrophilic controlled release polymers and one or more pharmaceutically acceptable excipients. The formulations provide extended release of morphine or salt thereof over a specified period of time after oral administration in humans or animals.

### FIELD OF THE INVENTION

The present invention relates to extended release matrix formulations of morphine or salts thereof and process of making such formulations.

### BACKGROUND OF THE INVENTION

It is well known in the pharmaceutical art to prepare formulations which provide extended release of pharmacologically active substances after oral administration to humans and animals. Extended release formulations decrease the frequency of administration required to maintain therapeutically effective plasma drug levels. In addition, by producing more constant blood levels, such formulations can reduce the large changes in plasma levels observed between doses. Extended release formulations are intended to provide a longer period of pharmacological action after administration than is ordinarily obtained after administration of immediate-release dosage form. Such longer periods of response provide therapeutic benefits that are not achieved by short acting, immediate release preparations. Further, extended release preparations result in better patient compliance resulting from the avoidance of missed doses through patient forgetfulness.

Opioids are mainly used for acute or chronic pain ranging from moderate to severe. All opioids have in common an unrivaled pain relieving efficacy without toxicity to the body. Morphine, oxymorphone, and hydromorphone are reserved for the upper-most region of the pain spectrum while moderately severe pain is often treated with oxycodone.

Morphine is an extremely powerful opiate analgesic drug and is the principal active agent in opium. Like other opioids, e.g. heroin, morphine acts directly on the central nervous system (CNS) to relieve pain. Orally, it is available as an elixir, concentrated solution, powder (for compounding) or in tablet form. Due to its poor oral bioavailability, oral morphine has only one-sixth to one-third of the potency of parenteral morphine. Morphine is also available in extended-release capsules for chronic administration, as well as immediate-release formulations. Morphine, which is considered to be prototypic opioid analgesic, has been formulated into 12 hour extended-release formulations (i.e., MS Contin® tablets, commercially available from Purdue Frederick Company).

Controlled release compositions of opioid analgesics such as morphine, hydromorphone or salts thereof are previously known in the art.

U.S. Pat. No. 5,520,931 discloses pH independent and zero order controlled release tablets of morphine that are coated with a water-insoluble diffusion membrane.

U.S. Pat. No. 5,952,005 discloses controlled release preparation containing particles having a core comprising a salt of morphine coated with a water-insoluble barrier layer.

U.S. Pat. No. 5,958,459 discloses dosage forms containing inert beads coated with an analgesic opioid followed by a controlled release overcoating layer.

U.S. Pat. No. 6,607,751 discloses controlled release formulations comprising combination of microbial polysaccharide and cellulose ether.

U.S. Pat. No. 6,251,430 discloses a sustained release tablet dosage form comprising a mixture of three different types of polymers: a water insoluble polymer; a pH dependent gelling polymer; and a pH-independent gelling polymer.

U.S. Pat. No. 6,399,096 discloses a solid, oral, controlled release pharmaceutical dosage form which comprises a pharmaceutical active ingredient dispersed in a controlled-release matrix. The matrix comprises a hydrophobic, fusible material having a melting point of greater than 40° C. and may also include material having a wicking agent which may be a hydrophilic, organic, polymeric, fusible substance or a particulate soluble or insoluble inorganic material.

U.S. Pat. No. 4,861,598 discloses controlled-release bases containing a combination of a higher aliphatic alcohol and an acrylic resin for the extended release of therapeutic agents. This patent teaches that the optimum control of drug release and a delay in retardation of generally 5-12 hours can be achieved by utilizing the matrix base in a range of 20-40% by weight of the total weight of the selected dosage unit. The patent further teaches that when using the acrylic resins in combination with the higher aliphatic alcohol there was unexpectedly a potentiation of the control of the drug release properties for the flow and controlled release of medicaments, particularly for highly water-soluble therapeutic agents.

U.S. Pat. Nos. 5,891,471 and 6,162,467 teaches process for preparing sustained-release particles composition comprising a hydrophobic and/or hydrophilic fusible carrier.

U.S. Pat. No. 4,990,341 discloses hydromorphone compositions wherein the dissolution rate in vitro of the dosage form, when measured by the USP Paddle Method at 100 rpm in 900 ml aqueous buffer (pH between 1.6 and 7.2) at 37° C., is between 12.5 and 42.5% (by wt) hydromorphone released after 1 hour, between 25 and 55% (by wt) released after 2 hours, between 45 and 75% (by wt) released after 4 hours and between 55 and 85% (by wt) released after 6 hours. The composition comprises at least one water soluble hydroxyalkyl cellulose and at least one digestible, long chain fatty aliphatic alcohol. This patent teaches that the ratio of the hydroxyalkyl cellulose and the aliphatic alcohol determines to a considerable extent the release of the active ingredient from the formulation.

Although these formulations are useful as sustained release compositions, there are known drawbacks to the above-described methods and compositions.

The above prior art teaches the use of combination of hydrophilic polymers along with aliphatic alcohols or acrylic acid polymers to achieve the desired release characteristics of the incorporated medicament in the gastrointestinal tract. The prior art further teaches that the combination of these hydrophilic polymers with aliphatic alcohols or acrylic acid polymers result in controlled-release of medicament with a pH-independent release profile. However, there are certain drawbacks associated with the use of these aliphatic alcohols. Higher aliphatic alcohols must be melted prior to being mixed with the cellulose polymer which results in energy consumption, messy clean-up and the need to use special equipment such as water-jacketed tanks.

### SUMMARY OF THE INVENTION

It is an object of the present invention to provide an extended release matrix formulation that is prepared easily, with lesser processing steps, lower energy consumption. Surprisingly, we have found that the hydrophilic polymer alone can be used as an extended release formulation component that gives the desired extended release for a water-soluble drug such as morphine, e.g., morphine sulfate. A hydrophilic matrix extended release system is a robust dynamic system composed of polymer wetting, hydration and dissolution. In such matrix systems, the hydrophilic polymer upon contact with water hydrates the outer surface to form a gel layer. The rate of diffusion of drug out of the gel layer and the rate of matrix erosion control the overall dissolution rate and drug delivery. Unlike prior art formulations, the formulations of the present invention are cost-effective, time-effective, less labor-intensive, and easy to manufacture on commercial scale without requiring complex processing steps.

According to one embodiment there are provided extended-release matrix formulations a therapeutically effective amount of morphine or salts thereof, one or more hydrophilic controlled-release polymers and one or more pharmaceutically acceptable excipients. The formulations provide extended release of morphine or salts thereof over a specified period of time after oral administration in humans or animals. The dissolution profile of such extended release formulations may be measured in vitro using the USP Basket (Type I) Method, at 100 rpm, in 900 ml aqueous buffer (pH 1.2 to 6.8), at 37±0.5° C.

According to another embodiment there are provided extended-release matrix formulations a therapeutically effective amount of morphine or salts thereof, hydroxypropyl methylcellulose having an apparent viscosity of 80,000-120,000 cP (2% in water at 20° C.) and one or more pharmaceutically acceptable excipients.

According to another embodiment there are provided extended release matrix formulations comprising therapeutically effective amount of morphine or salts thereof, 20-40 mg of hydroxypropyl methylcellulose having an apparent viscosity of 80,000-120,000 cP (2% in water at 20° C.) and one or more pharmaceutically acceptable excipients.

According to still another embodiment there are provided processes for preparing the extended release matrix formulations of morphine or salts thereof.

### DETAILED DESCRIPTION OF THE INVENTION

The extended release matrix formulations comprise morphine or salts thereof, one or more hydrophilic controlled-release polymers and one or more pharmaceutically acceptable excipients such that when administered orally the formulations release morphine or salts thereof in an extended release manner over a prolonged period of time.

According to one embodiment, the formulations prepared show an in vitro dissolution profile of morphine or salts thereof, when measured using USP I Method, at 100 rpm, in 900 ml aqueous buffer (pH 1.2 to 6.8), at 37±0.5° C., to be between 20% and 45% released after 1 hour, between 30% and 65% released after 2 hours, between 60% and 90% released after 4 hours, and between 70% and 100% released after 6 hours. The formulation releases morphine in an extended manner, thereby avoiding dose dumping upon oral administration. Surprisingly, this aspect could be achieved with the use of hydrophilic controlled release polymers alone. Thus, the formulations can consist essentially of the materials described above.

USP I Method is the Basket Method described, e.g., in U.S. Pharmacopoeia XXV (2002), page no: 2011-2012.

The extended release formulation may contain morphine in the range of between 1 to 500 mg, e.g., between about 15 to 250 mg. Morphine is preferably present in an amount suitable for twice daily dosing. The formulation may contain morphine or salts thereof, e.g., morphine sulfate.

The “hydrophilic controlled0-release polymer” may be selected, for example, from one or more of cellulose derivatives selected from hydroxypropyl methylcellulose, hydroxyethyl cellulose, hydroxypropyl cellulose, carboxymethyl cellulose, sodium carboxymethyl cellulose; and gums selected from xanthan gum, karaya gum, locust bean gum, alginic acid and sodium alginate. The hydroxypropyl methylcellulose may be, for example, the commercially available products such as Methocel® premium product grades having specific apparent viscosities, e.g., viscosities ranging from about 100-150,000 cP (2% in water at 20° C.) such as K100, K4M, K15M, K100M, E4M, E10M; viscosities ranging from 80000-120,000 cP (2% in water at 20° C.) such as Methocel K100M CR. Surprisingly, it was observed that the amount of the hydrophilic controlled-release polymer per unit dose of morphine or salt thereof plays a major role in the release characteristic of the formulation. The amount of hydrophilic controlled-release polymer may range from about 20-40 mg per unit dose of morphine or salt thereof e.g. the amount may range from about 22-30 mg per unit dose of morphine or salt thereof. The formulations may contain other release-retarding polymers along with the hydrophilic polymers. However, hydrophilic polymers alone can be used to obtain the extended-release formulations with desirable characteristics of the invention. Thus, the extended-release formulation can consist essentially of morphine or salts thereof, one or more hydrophilic controlled-release polymers and one or more pharmaceutically acceptable excipients.

The extended release formulation may also contain “pharmaceutically acceptable excipients” selected from, for example, one or more of diluents, binders, lubricants and glidants.

The diluent may, for example, be selected from, for example, one or more of microcrystalline cellulose, lactose, dicalcium phosphate and starch.

The binder may be selected from, for example, one or more of starch, polyvinylpyrrolidone, natural or synthetic gum and cellulosic polymers.

The lubricants and glidants may be selected from, for example, one or more of talc, colloidal silicon dioxide and magnesium stearate.

The extended-release formulation of morphine may be obtained in the form of tablet, bead, pellet or capsule. The tablet may be uncoated tablet, coated tablet, or minitablets e.g. the extended-release formulation may be a matrix tablet with or without a non-functional coating.

The tablet may be prepared by wet granulation, dry granulation/slugging methods or direct compression processes.

According to one embodiment, the extended-release matrix formulation of morphine of the present invention is bioequivalent to the branded formulation.

The term “branded formulation” as used herein refers to tablet formulation of morphine sulfate, commercially available in U.S. as MS Contin® tablets, from Purdue Frederick Company.

The following non-limiting examples further illustrate the extended-release formulations of morphine or salt thereof, and process of making such formulations.

### EXAMPLES 1-5

| | Quantity (mg/Tablet) | | | | |
|---|---|---|---|---|---|
| | Example no: | | | | |
| Ingredient | 1 | 2 | 3 | 4 | 5 |
| Morphine | 60.0 | 15.0 | 30.0 | 100.0 | 200.0 |
| Sulfate•Pentahydrate | | | | | |
| Hydroxypropyl | 27.0 | 29.65 | 27.0 | 27.0 | 27.0 |
| methylcellulose | | | | | |
| Lactose monohydrate | 52.5 | 30.62 | 42.5 | 62.5 | 108.7 |
| Povidone | 4.5 | 2.45 | 3.25 | 6.30 | 10.90 |
| Isopropyl alcohol* | q.s. | q.s. | q.s. | q.s. | q.s. |
| Colloidal silicon dioxide | 1.75 | 0.940 | 1.250 | 2.350 | 4.200 |
| Stearic Acid | 1.75 | 0.94 | 1.25 | 2.35 | 4.200 |
| Magnesium Stearate | 2.5 | 1.40 | 1.75 | 3.50 | 6.0 |
| Purified water* | q.s. | q.s. | q.s. | q.s. | q.s. |
| Compression Weight | 150.0 | 81.0 | 107.0 | 204.0 | 361.0 |
| Opadry | q.s. | q.s. | q.s. | q.s. | q.s. |
| Purified water* | q.s. | q.s. | q.s. | q.s. | q.s. |

*Lost during processing

### Brief Manufacturing Procedure

1. All ingredients were accurately weighed.
2. Morphine sulfate, Hydroxypropyl methylcellulose and Lactose monohydrate were sifted through a suitable mesh and mixed in Rapid mixer granulator (RMG).
3. The solution of Povidone in isopropyl alcohol was prepared.
4. Blend of Step 2 was granulated with solution of Step 3.
5. The granules obtained in step 4 were dried in fluid bed drier (FBD) to remove isopropyl alcohol.
6. The dried granules were sized through sieve #30.
7. Colloidal silicon dioxide was sifted through sieve #30 and Stearic acid was sifted through sieve #40.
8. Blend of Step 7 was mixed with granules of Step 6 in a V-blender.
9. Magnesium stearate was sifted through sieve #40 and then mixed with the blend of Step 8 in a V-blender to obtain a final blend.
10. The final blend was compressed into tablets using suitable toolings.
11. The tablets were then coated using the dispersion of Opadry in purified water to achieve a desired weight build up.

The in vitro release profile of morphine from formulations given in examples 1-5, measured by the method described herein (USP I, 900 ml, 100 rpm), is given below.

### EXAMPLE 1

| | % release of morphine | | | |
|---|---|---|---|---|
| | pH 1.2 | | pH 6.8 | |
| Time | SGF (without | pH 4.5 acetate | phosphate | |
| (hrs) | enzymes) | buffer | buffer | Water |
| 0 | 0 | 0 | 0 | 0 |
| 0.5 | 24 | 20 | 20 | 17 |
| 1 | 38 | 32 | 31 | 29 |
| 2 | 59 | 52 | 50 | 47 |
| 3 | 74 | 66 | 64 | 62 |
| 4 | 86 | 78 | 75 | 74 |
| 6 | 97 | 94 | 91 | 92 |
| 8 | 100 | 99 | 98 | 100 |
| 10 | 100 | 101 | 101 | 101 |
| 12 | 100 | 100 | 102 | 101 |

### EXAMPLES 2-5

| | | | | |
|---|---|---|---|---|
| Time (hrs) | Example 2 | Example 3 | Example 4 | Example 5 |

% release of morphine
pH 4.5 acetate buffer

| | | | | |
|---|---|---|---|---|
| 0 | 0 | 0 | 0 | 0 |
| 0.5 | 26 | 23 | 17 | 18 |
| 1 | 40 | 32 | 29 | 29 |
| 2 | 62 | 56 | 47 | 48 |
| 3 | 77 | 71 | 60 | 62 |
| 4 | 88 | 83 | 73 | 76 |
| 6 | 100 | 97 | 91 | 97 |
| 8 | 102 | 102 | 102 | 106 |
| 10 | 104 | 103 | 105 | 106 |
| 12 | 105 | 103 | 107 | 107 |

% release of morphine
pH 6.8 phosphate buffer

| | | | | |
|---|---|---|---|---|
| 0 | 0 | 0 | 0 | 0 |
| 0.5 | 24 | 22 | 19 | 20 |
| 1 | 37 | 36 | 30 | 30 |
| 2 | 57 | 54 | 47 | 47 |
| 3 | 71 | 68 | 59 | 60 |
| 4 | 82 | 80 | 70 | 72 |
| 6 | 94 | 94 | 87 | 91 |
| 8 | 99 | 101 | 97 | 101 |
| 10 | 100 | 103 | 101 | 104 |
| 12 | 101 | 104 | 104 | 105 |

% release of morphine
Water

| | | | | |
|---|---|---|---|---|
| 0 | 0 | 0 | 0 | 0 |
| 0.5 | 25 | 20 | 16 | 19 |
| 1 | 38 | 32 | 26 | 29 |
| 2 | 57 | 51 | 43 | 47 |
| 3 | 72 | 64 | 55 | 61 |
| 4 | 82 | 76 | 68 | 74 |
| 6 | 95 | 92 | 86 | 95 |
| 8 | 99 | 97 | 98 | 103 |
| 10 | 99 | 99 | 102 | 104 |
| 12 | 99 | 99 | 104 | 104 |

### EXAMPLES 6-7

Quantity
(mg/Tablet)
Example
no:

| Ingredients | 6 | 7 |
|---|---|---|
| Morphine Sulfate•Pentahydrate | 60.0 | 60.0 |
| Hydroxypropyl methylcellulose | 22.5 | 30.0 |
| Lactose monohydrate | 35.0 | 27.5 |
| Povidone | 4.5 | 4.5 |
| Isopropyl alcohol* | q.s. | q.s. |
| Colloidal silicon dioxide | 1.5 | 1.5 |
| Stearic Acid | 1.5 | 1.5 |
| Magnesium Stearate | 2.0 | 2.0 |
| Compression Weight | 127.0 | 127.0 |
| Opadry | q.s. | q.s. |
| Purified water* | q.s. | q.s. |

*Lost during processing

### Brief Manufacturing Procedure

1. All ingredients were accurately weighed.
2. Morphine sulfate, Hydroxypropyl methylcellulose and Lactose monohydrate were sifted through a suitable mesh and mixed in Rapid mixer granulator (RMG).
3. The solution of Povidone in isopropyl alcohol was prepared.
4. Blend of Step 2 was granulated with solution of Step 3.
5. Then granules obtained in step 4 were dried in fluid bed drier (FBD) to remove isopropyl alcohol.
6. The dried granules were sized through sieve #30.
7. Colloidal silicon dioxide was sifted through sieve #30 and Stearic acid was sifted through sieve #40.
8. Blend of Step 7 was mixed with granules of Step 6 in a V-blender.
9. Magnesium stearate was sifted through sieve #40 and then mixed with the blend of Step 8 in a V-blender to obtain the final blend.
10. The final blend was compressed into tablets using suitable toolings.
11. The tablets were then coated using the dispersion of Opadry in purified water to achieve a desired weight build up.

The in vitro release profile of morphine from formulations given in example 6-7, measured by the method described herein (USP I, 900 ml, 100 rpm), is given below.

### EXAMPLE 6

| | % release of morphine | | | |
|---|---|---|---|---|
| | pH 1.2 | | pH 6.8 | |
| Time | SGF (without | pH 4.5 acetate | phosphate | |
| (hrs) | enzymes) | buffer | buffer | Water |
| 0 | 0 | 0 | 0 | 0 |
| 0.5 | 25 | 20 | 20 | 19 |
| 1 | 39 | 32 | 31 | 31 |
| 2 | 61 | 53 | 49 | 50 |
| 3 | 77 | 67 | 62 | 65 |
| 4 | 88 | 79 | 74 | 78 |
| 6 | 97 | 94 | 88 | 92 |
| 8 | 99 | 99 | 95 | 98 |
| 10 | 100 | 100 | 97 | 100 |
| 12 | 101 | 100 | 99 | 100 |

### EXAMPLE 7

| | % release of morphine | | | |
|---|---|---|---|---|
| | pH 1.2 | | pH 6.8 | |
| Time | SGF (without | pH 4.5 acetate | phosphate | |
| (hrs) | enzymes) | buffer | buffer | Water |
| 0 | 0 | 0 | 0 | 0 |
| 0.5 | 23 | 19 | 18 | 18 |
| 1 | 37 | 31 | 28 | 29 |
| 2 | 58 | 50 | 46 | 47 |
| 3 | 74 | 64 | 59 | 61 |
| 4 | 87 | 77 | 71 | 73 |
| 6 | 101 | 94 | 88 | 91 |
| 8 | 104 | 101 | 98 | 101 |
| 10 | 105 | 102 | 102 | 105 |
| 12 | 106 | 103 | 104 | 105 |

### Bioequivalence Studies

### Pharmacokinetic Study Design:

Products evaluated

| | |
|---|---|
| Test (A): | Morphine sulfate ER tablets 60 mg |
| | Manufactured by Ranbaxy Research Lab. Ltd., India |
| Test (B): | Morphine sulfate ER tablets 60 mg |
| | Manufactured by Ranbaxy Research Lab. Ltd., India |
| Reference (C): | MS Contin ® 60 mg ER tablets (Lot no.: YF14) |
| | Manufactured by Purdue Frederick Co., USA |
| Treatments*: | A: Single oral dose of Test product A (Fed) |
| | B: Single oral dose of Test product B (Fed) |
| | C: Single oral dose of Reference product (Fed) |

*Number of subjects (Human volunteers) = 18 in each case.

Summary Statistics of Bioequivalence Studies*

| | | | | |
|---|---|---|---|---|
| | | AUC₀₋ₜ | AUC_{0-∞} | |
| Product/Statistics | Cₘₐₓ (ng/mL) | (ng · h/mL) | (ng · h/mL) | Tₘₐₓ (h) |

Product A

| | | | | |
|---|---|---|---|---|
| Mean | 36.35 | 241.84 | 254.14 | 2.98 |
| CV (%) | 11.44 | 69.57 | 69.28 | 1.40 |

Product B

| | | | | |
|---|---|---|---|---|
| Mean | 39.44 | 250.39 | 261.55 | 3.95 |
| CV (%) | 14.48 | 76.71 | 76.12 | 0.88 |

Product C

| | | | | |
|---|---|---|---|---|
| Mean | 39.45 | 253.29 | 267.64 | 3.59 |
| CV (%) | 12.58 | 83.95 | 83.69 | 1.42 |

10/23 Ratio of least squares mean

| | | | | |
|---|---|---|---|---|
| A/C (%) | 92.77 | 97.26 | 96.36 | — |

90% Confidence intervals (A/C)

| | | | | |
|---|---|---|---|---|
| Lower limit | 83.34 | 93.36 | 92.57 | — |
| Upper limit | 103.27 | 100.69 | 100.3 | — |

Ratio of least squares mean

| | | | | |
|---|---|---|---|---|
| B/C (%) | 98.86 | 100.09 | 98.70 | — |

90% Confidence intervals (B/C)

| | | | | |
|---|---|---|---|---|
| Lower limit | 88.81 | 96.69 | 94.8 | — |
| Upper limit | 110.04 | 103.61 | 102.73 | — |

*Number of subjects (Human volunteers) = 18 in each case.

As evident from the above pharmacokinetic data, extended-release matrix formulations of Morphine as per the present invention are bioequivalent to the branded formulation.

We claim:

1. An extended-release matrix formulation consisting essentially of a therapeutically effective amount of morphine or salts thereof, one or more hydrophilic controlled-release polymers and one or more pharmaceutically acceptable excipients.

2. The extended-release matrix formulation according to claim 1, wherein the hydrophilic controlled-release polymer is selected from one or more of cellulose derivatives and gums.

3. The extended-release matrix formulation according to claim 2, wherein the cellulose derivative is selected from one or more of hydroxypropyl methylcellulose, hydroxyethyl cellulose, hydroxypropyl cellulose, carboxymethyl cellulose and sodium carboxymethyl cellulose.

4. The extended-release formulation according to claim 2, wherein the gum is selected from one or more of xanthan gum, karaya gum, locust bean gum, alginic acid and sodium alginate.

5. The extended-release matrix formulation according to claim 3, wherein the hydroxypropyl methylcellulose has an apparent viscosity in the range of about 100-1, 50,000 cP (2% in water at 20° C.).

6. The extended-release matrix formulation according to claim 5, wherein the hydroxypropyl methylcellulose has an apparent viscosity in the range of about 80,000-120,000 cP (2% in water at 20° C.).

7. The extended-release matrix formulation according to claim 6, wherein the hydroxypropyl methylcellulose is present in 20-40 mg per unit dose of morphine or salts thereof.

8. The extended-release matrix formulation according to claim 1, wherein the pharmaceutically acceptable excipients are selected from one or more of diluents, binders, lubricants and glidants.

9. The extended-release matrix formulation according to claim 8, wherein the diluent is selected from one or more of microcrystalline cellulose, lactose, dicalcium phosphate and starch.

10. The extended-release matrix formulation according to claim 8, wherein the binder is selected from one or more of starch, polyvinylpyrrolidone, natural or synthetic gum and cellulosic polymers.

11. The extended release matrix formulation according to claim 1, wherein the dissolution profile in vitro, when measured using USP I Method, at 100 rpm, in 900 ml aqueous buffer (pH 1.2 to 6.8), at 37±0.5° C. is: between 20% and 45% released after 1 hour, between 30% and 65% released after 2 hours, between 60% and 90% released after 4 hours, and between 70% and 100% released after 6 hours.
